(19) United States
(12) Patent Application Publication
(10) Pub. No.: US 2008/0288015 A1
(43) Pub. Date: Nov. 20, 2008

(54) Diaphragm stimulation device and method for use with cardiovascular or heart patients

(75) Inventors: Amir J. Tehrani, Los Altos, CA (US); Rose Province, San Jose, CA (US); Chang Lee, Redwood City, CA (US); David Ligon, San Francisco, CA (US)

Correspondence Address: LEVINE BAGADE HAN LLP, 2483 EAST BAYSHORE ROAD, SUITE 100, PALO ALTO, CA, 94303, US

(73) Assignee: RMX, LLC, San Francisco, CA (US)

(21) Appl. No.: 12/150,052

(22) Filed: Apr. 23, 2008

Related U.S. Application Data
(63) Continuation-in-part of application No. 12/082,057, filed on Apr. 8, 2008.
Application No. 12/082,057 is a continuation-in-part of application No. 12/069,823, filed on Feb. 13, 2008.
Application No. 12/069,823 is a continuation-in-part of application No. 12/004,932, filed on Dec. 21, 2007.
Application No. 12/004,932 is a continuation-in-part of application No. 11/981,342, filed on Oct. 31, 2007.
Application No. 11/981,342 is a continuation-in-part of application No. 11/480,074, filed on Jun. 29, 2006.
Application No. 11/480,074 is a continuation-in-part of application No. 11/271,726, filed on Nov. 10, 2005.
Application No. 11/271,726 is a continuation-in-part of application No. 10/966,484, filed on Oct. 15, 2004.
Application No. 10/966,484 is a continuation-in-part of application No. 10/966,474, filed on Oct. 15, 2004.
Application No. 10/966,474 is a continuation-in-part of application No. 10/966,421, filed on Oct. 15, 2004.
Application No. 10/966,421 is a continuation-in-part of application No. 10/966,472, filed on Oct. 15, 2004.
Application No. 10/966,472 is a continuation-in-part of application No. 10/686,891, filed on Oct. 15, 2003.
(60) Provisional application No. 60/931,205, filed on May 22, 2007.

(51) Int. Cl. A61N 1/36

(52) U.S. Cl. 607/42

(57) ABSTRACT

A method for treating a patient is provided where a sensed cardiac rhythm is used to adjust electrical stimulation that is used to activate a diaphragm to cause contraction. A cardiac rhythm management device in combination with a diaphragm stimulation device is also provided which includes a cardiac stimulation device configured to provide stimulation to control cardiac rhythm of a subject; and an electrical stimulation device configured to provided electrical stimulation to a subject to activate a diaphragm to increase functional residual capacity of the subject.

This application claims priority of Provisional Application No. 60/931,205 and; and is a continuation in part of U.S. application Ser. No. 12/082,057 entitled: DEVICE AND METHOD FOR TREATING CARDIOVASCULAR DISORDERS OF THE CARDIOVASCULAR SYSTEM OR HEART filed Apr. 8, 2008; and is a continuation in part U.S. application Ser. No. 12/069,823 filed Feb. 13, 2008, and of U.S. application Ser. No. 12/004,932 filed Dec. 21, 2007; and of U.S. application Ser. No. 11/981,342 filed Oct. 31, 2007; and of U.S. application Ser. No. 11/480,074 filed Jun. 29, 2006 and of U.S. application Ser. No. 11/271,315 filed Nov. 10, 2005; and of U.S. application Ser. No. 11/271,554 filed Nov. 10, 2005; and of U.S. application Ser. No. 11/271,353 filed Nov. 10, 2005; and of U.S. application Ser. No. 11/271,264 filed Nov. 10, 2005; and of U.S. patent application Ser. No. 10/966,487 filed Oct. 15, 2004; and of U.S. application Ser. No. 11/480,074 filed Jun. 29, 2006 which is a continuation in part of U.S. application Ser. No. 11/271,726 filed Nov. 10, 2005 which is a continuation in part of U.S. application Ser. No. 10/966,484 filed Oct. 15, 2004; U.S. application Ser. No. 10/966,474, filed Oct. 15, 2004; U.S. application Ser. No. 10/966,421, filed Oct. 15, 2004; and U.S. application Ser. No. 10/966,472 filed Oct. 15, 2004 which are continuations in part of U.S. application Ser. No. 10/686,891 filed Oct. 15, 2003 entitled: BREATHING DISORDER DETECTION AND THERAPY DELIVERY DEVICE AND METHOD all of which are incorporated in their entirety herein by reference without limitation.

### FIELD OF THE INVENTION

The present invention relates to a device and method for providing diaphragm stimulation in cardiovascular and heart failure (or dysfunction) patients.

### BACKGROUND OF THE INVENTION

In copending Application entitled DEVICE AND METHOD FOR TREATING DISORDERS OF THE CARDIOVASCULAR SYSTEM OR HEART, filed on even date herewith and incorporated in its entirety herein by reference without limitation, electrical stimulation is described to elicit a diaphragm response to provide therapy to patients with heart failure, heart dysfunction or other cardiovascular related disease. Such stimulation may be provided to treat heart failure/dysfunction or other cardiovascular disease and/or may be provided to treat disordered breathing that frequently is associated with heart failure. Such patients may be using a cardiac rhythm management device such as a pacemaker, CRT or ICD or may at some time be a candidate for such device. Accordingly, it would be desirable to provide a device that provides stimulation to elicit a diaphragm response that may work in concert with one or more such devices.

It would further be desirable to provide a device that may control the diaphragm stimulation in response to one or more cardiac conditions.

### SUMMARY OF THE INVENTION

In accordance with one aspect of the invention, a diaphragm stimulation device, i.e., configured to stimulate tissue to elicit a diaphragm response, is provided with an cardiac electrogram sensor. The diaphragm stimulator is configured to control or adjust stimulation in response to cardiac electrogram sensed by the cardiac electrogram sensor. The sensed cardiac electrogram may provide information on a cardiac condition or event and may control stimulation to avoid device-device interaction at the occurrence of a cardiac event. According to one variation, stimulation may be turned off when a cardiac event or condition is present for purposes of safety and to allow other intervention. The stimulator may also provide or adjust stimulation based on a cardiac event or condition, to therapeutically benefit the patient during such event or condition.

In accordance with one aspect of the invention, stimulation is provided to the diaphragm or phrenic nerve to elicit a diaphragm response to thereby provide a therapeutic effect for a heart failure or other cardiac or cardiovascular patient.

In accordance with one aspect of the invention, stimulation to elicit a diaphragm response is provided to increase or normalize lung volume and in particular to increase functional residual capacity. It is believed that stimulation to increase or to normalize lung volume or functional residual capacity may have one or more effects that may be therapeutic to cardiovascular or heart failure patients. Normalizing herein may include for example, bringing a physiological parameter into a normal or healthy region for patients or for a particular patient, or to a level appropriate for a condition or state of a patient.

In accordance with another aspect of the invention stimulation is provided to control breathing to reduce respiration rate and thereby reduce hypertension, reduce sympathetic nerve bias, and/or provide improved blood gas levels.

In accordance with another aspect of the invention stimulation is provided to control minute ventilation to therapeutically effect blood gas levels.

In accordance with another aspect of the invention, stimulation is provided to create a deep inspiration or an increased tidal volume to thereby reduce sympathetic nerve bias, improve blood gas levels, stimulate reflexes for example the Hering-Bruer reflex related to activating stretch receptors, increase lung volume, normalize or reset breathing or provide other beneficial therapies to improve cardiovascular function or heart failure condition.

In accordance with another aspect of the invention stimulation may be provided to manipulate intrathoracic pressure to thereby produce a therapeutic effect. According to one embodiment, stimulation is provided to reduce intrathoracic pressure to thereby increase ventricular filling.

In accordance with another aspect of the invention stimulation is provided to reduce breathing disorders to thereby improve condition of a heart failure patient.

In accordance with another aspect of the invention a combined cardiac rhythm management device and diaphragm/phrenic nerve stimulation device is provided to provide an enhanced combined treatment device.

In accordance with another aspect of the invention the stimulation device may be used to treat one or more diseases, disorders and conditions that may relate to, have co-morbidities with, affect, be affected by respiratory or lung health status, respiration, ventilation, or blood gas levels. Such diseases and disorders may include but are not limited to obstructive respiratory disorders, restrictive respiratory disorders, vascular respiratory disorders, upper airway resistance syndrome, snoring, obstructive apnea; central respiratory disorders, central apnea; hypopnea, hypoventilation, obesity hypoventilation syndrome other respiratory insufficiencies, inadequate ventilation or gas exchange, chronic obstructive pulmonary diseases; asthma; emphysema; chronic bronchitis; circulatory disorders; hemodynamic disorders; hypertension; heart disease; chronic heart failure; cardiac rhythm disorders; neurodegenerative disorders; ALS; MS; obesity or injuries in particular affecting breathing or ventilation.

These and other aspects of the invention are set forth herein in the abstract, specification and claims.

### DESCRIPTION OF THE DRAWINGS

FIG. 1 is a front schematic view illustrating a device in accordance with an aspect of the invention.

FIG. 2A, illustrates a composite sensed signal including diaphragm EMG and CARDIAC ELECTROGRAM.

FIG. 2B illustrates a diaphragm EMG signal filtered from the composite signal of FIG. 2A.

FIG. 2C illustrates and CARDIAC ELECTROGRAM signal filtered from the composite signal of FIG. 2A.

FIGS. 3A, 3B, and 3C illustrate respectively, an CARDIAC ELECTROGRAM signal in which an arrhythmia event occurs, a schematic illustration of lung volume and a therapeutic stimulation signal configured elicit a diaphragm response.

FIGS. 4A, 4B, and 4C illustrate respectively, an CARDIAC ELECTROGRAM signal in which an arrhythmia event occurs, a schematic illustration of lung volume and a therapeutic stimulation signal configured elicit a diaphragm response.

FIG. 5 is a flow chart illustrating an example of use of a method and device in accordance with the invention.

### DETAILED DESCRIPTION

Examples of various devices for and uses of diaphragm stimulation are described in related applications set forth above and in copending patent application entitled: DEVICE AND METHOD FOR TREATING DISORDERS OF THE CARDIOVASCULAR SYSTEM OR HEART, filed on even date herewith, all of which are incorporated herein by reference without limitation. A diaphragm stimulator in accordance with the invention also includes a cardiac electrogram sensor which may be used in conjunction with any diaphragm stimulation therapy.

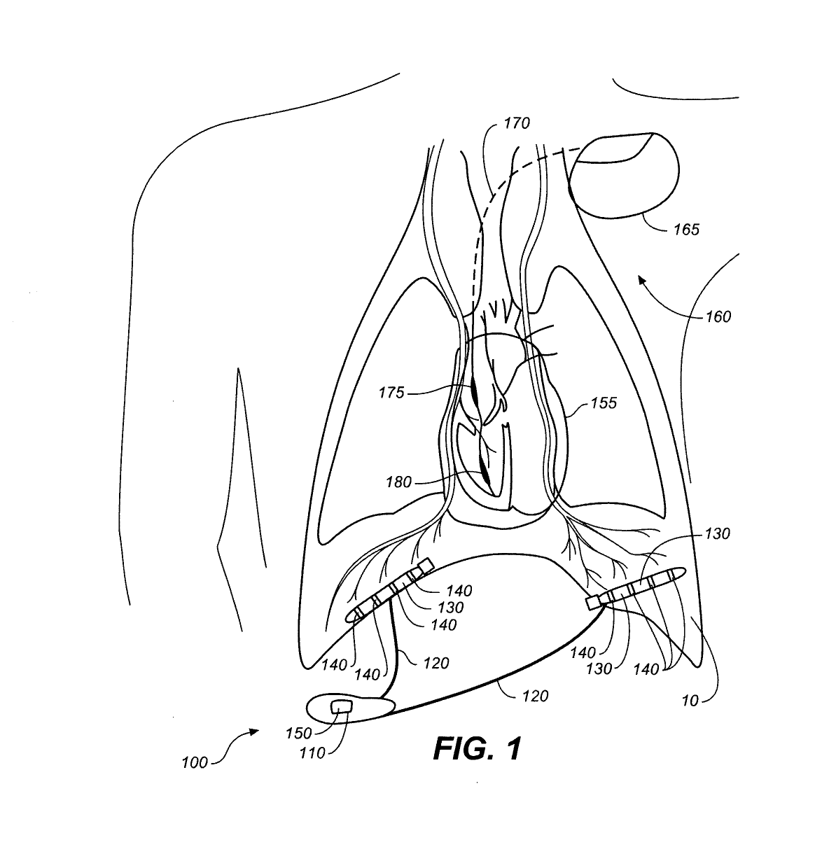

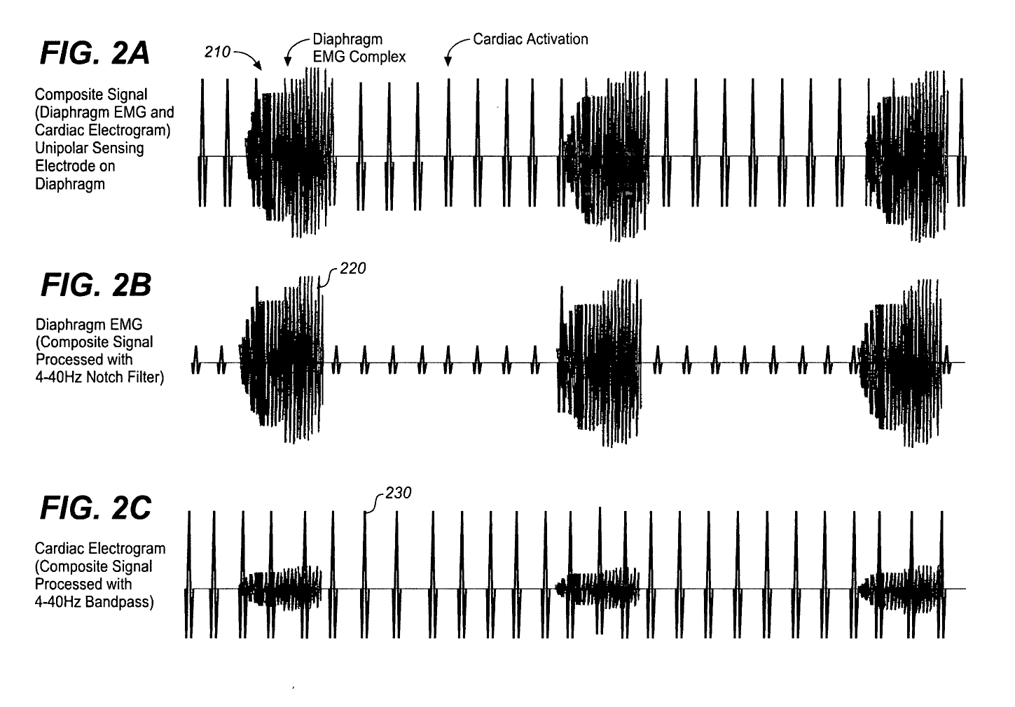

FIG. 1 illustrates a diaphragm stimulator 100 in accordance with the invention positioned on the diaphragm 10. The stimulator may be positioned from an abdominal approach or a thoracoscopic approach as described, for example, one or patent applications set forth herein. The diaphragm stimulator 100 includes an implantable pulse generator 110 and leads 120 extending to electrode assemblies 130 positioned on the hemidiaphragms 20. The electrode assemblies 130 include at least one electrode 140 positionable on the diaphragm 10. The electrode assemblies 130 are coupled to a signal processor 150, e.g., located with the pulse generator 110, configured to process a composite signal, for example as shown in FIG. 2A to obtain a diaphragm EMG signal and/or an cardiac electrogram as shown in FIGS. 2B and 2C respectively. The stimulator 100 may further be programmed to control stimulation in response to cardiac electrogram information sensed by one or more electrodes 140. The electrode assembly 130 includes an electrode 135 that acts as a stimulation electrode as well as a sensor for sensing EMG and cardiac electrogram. The cardiac electrogram sensor described herein includes the electrode 135 and signal processing, for example as described herein. The EMG sensor and/or the cardiac electrogram sensor may be separate from the electrode 135 as well. Examples of programmable diaphragm are set forth in one or more of the related patent applications set forth herein.

FIG. 1 further illustrates a CRM device 160 implanted with leads in the heart 155. The CRM device comprises a subcutaneously implanted pulse generator 165 with intravenous lead 170 extending into heart and terminating in atrial electrode 175 and ventricular electrode 180. The diaphragm stimulator 100 is configured to avoid device-device interaction with CRM device 160. For example, defibrillation therapy produces a strong electric field in the body that may be sensed by at least one 140 electrode on the diaphragm. The electrode 140 may be arranged, for example, with a far reference such that it more effectively picks up a global signal such as the defibrillation therapy as sensed at the diaphragm. When the diaphragm stimulation device senses that defibrillation is occurring stimulation is adjusted or turned off to allow for defibrillation without interference from diaphragm stimulation, and to lower the risk of noise detection which may interfere with cardiac therapies. Antitachycardia pacing which treats lower rate tachycardias may also be detected by the diaphragm stimulation device, and trigger a halting or change of diaphragm based therapy.

According to another aspect of the invention, the CRM device 160 is configured to avoid negative device/device interaction with a diaphragm stimulator 100. Diaphragm stimulation produces a characteristic electric field in the body that may be sensed by a sensing electrode component of a CRM device, for example, based on a known stimulation signal characteristics such as frequency, amplitude and/or duration of the stimulation. The CRM device may be programmed to recognize one or more of these characteristics. Programming and detection may occur, for example, when the diaphragm stimulation device is implanted so that the precise characteristics of the actual stimulation as seen by the CRM device can be recognized. The sensing electrode of the CRM device 160 may be arranged, for example, with a far reference such that it more effectively picks up a global signal such as the diaphragm stimulation. If a CRM device senses that diaphragm stimulation is occurring then it may be configured to adjust it's sense algorithms for reduced sensitivity to a diaphragm stimulation artifact. In accordance with another aspect of the invention, the CRM device may be configured as a defibrillator where the defibrillator is configured to avoid entering a noise detection state during a time in which diaphragm stimulation is occurring. Often defibrillation therapy is not allowed during noise detection. Accordingly turning off noise detection during diaphragm stimulation permits pacing or defibrillation to occur during diaphragm stimulation.

While FIG. 1 illustrates a diaphragm stimulator positioned in a specific location, a diaphragm stimulator in accordance one or more aspects of the invention may be positioned in other locations, for example on the phrenic nerve, subcutaneously, transvenously or externally. Such stimulator may include a stimulation electrode and an cardiac electrogram sensor and may be programmed in accordance with the invention to control stimulation based on sensed cardiac electrogram information.

FIG. 2A illustrates a composite signal 210 including a diaphragm EMG and an cardiac electrogram sensed at one or more electrodes of a diaphragm stimulator in accordance with the invention. This signal 210 as illustrated is representative of a diaphragm EMG and cardiac electrogram sensed at a stimulator configured to be positioned on the diaphragm of a subject. FIG. 2B illustrates a signal 220 comprising an EMG signal 220 processed from the signal 210 of FIG. 2A where the signal has been processed through a 4-40 Hz notch filter. FIG. 2C illustrates a signal 230 comprising an ECG signal 230 processed through a 4-40 Hz bandpass filter.

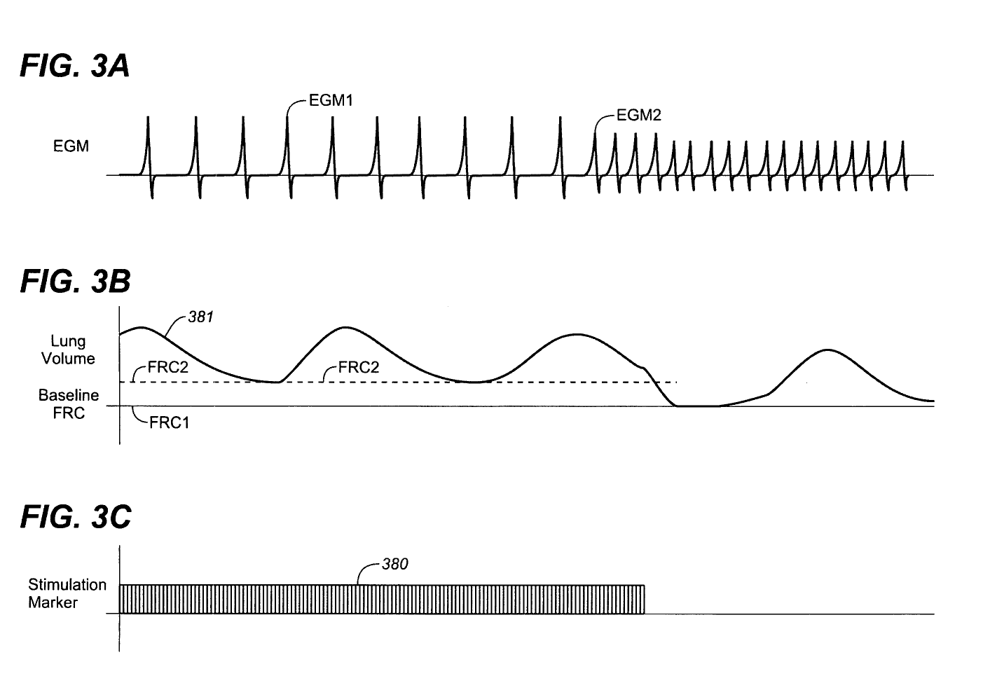

FIGS. 3A-3C illustrate a device and method in accordance with an aspect of the invention. FIG. 3C schematically illustrates a stimulation signal 380 to stimulate tissue to elicit a diaphragm response. The signal 380 is configured to increase functional residual capacity. Stimulation such as a bias stimulation to increase FRC is described for example in one or more related patent applications set forth herein. As illustrated by the lung volume 381 in FIG. 3B, the stimulation in FIG. 3C causes a functional residual capacity FRC2 which is greater than a baseline functional residual capacity FRC1. The cardiac electrogram (EGM), EGM1 during stimulation signal 380, is normal. EGM2 indicates an arrhythmia. The sensor that senses cardiac electrogram may also be configured to identify an irregular cardiac electrogram, such as, for example, an arrhythmia. The stimulation signal 380 is turned off by a controller when the cardiac electrogram signal is identified as an irregular cardiac electrogram.

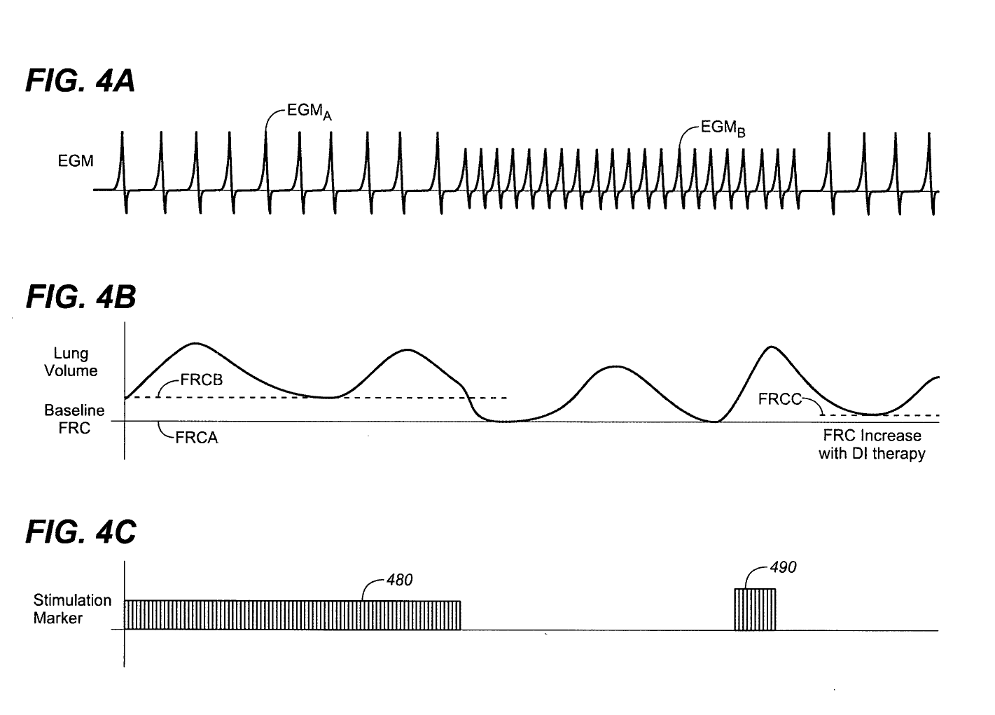

FIGS. 4A-4C illustrate a device and method in accordance with an aspect of the invention. FIG. 4C schematically illustrates a stimulation signal 480 to stimulate tissue to elicit a diaphragm response. The signal 480 is configured to increase functional residual capacity (FRC). Stimulation such as a bias stimulation to increase FRC is described for example in one or more related patent applications set forth herein. As illustrated by the lung volume in FIG. 4B, the stimulation in FIG. 4C causes a functional residual capacity FRC_{B} which is greater than a baseline functional residual capacity FRC_{A}. The cardiac electrogram, EGM_{A} during stimulation signal 480, is normal. EGM_{B} indicates an arrhythmia. The sensor that senses cardiac electrogram may also be configured to identify an irregular cardiac electrogram, such as, for example, an arrhythmia. The stimulation signal 480 is turned off by a controller when the cardiac electrogram signal is identified as an irregular cardiac electrogram. A stimulation signal 490 is delivered to stimulate a deep inspiration or a breath with an increased tidal volume when the controller identifies the irregular cardiac electrogram, EGM_{B} as an arrhythmia. An increase in FRC to FRC_{c} is shown resulting from deep inspiration therapy from stimulation signal 490.

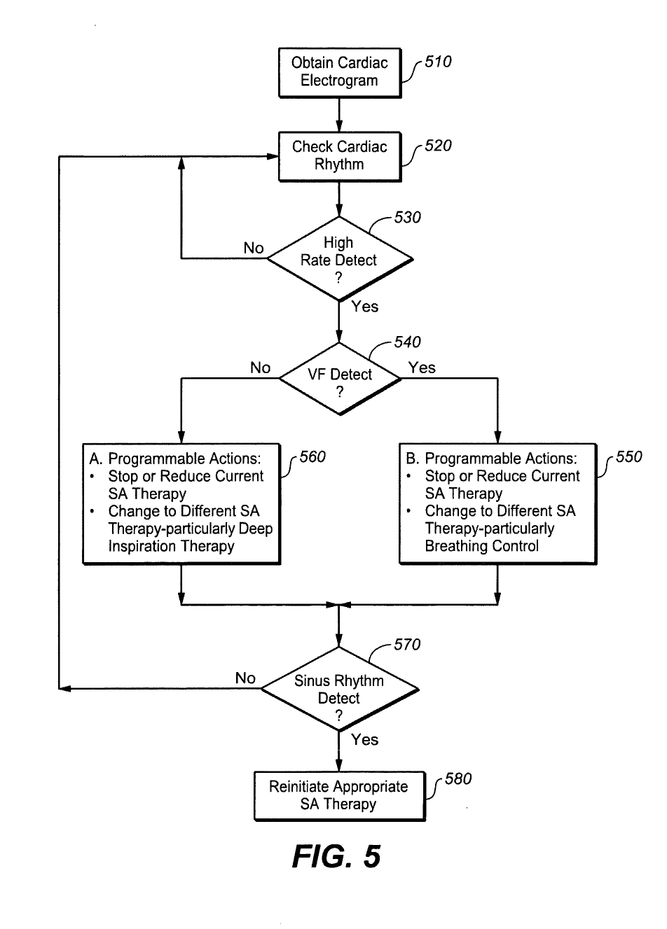

FIG. 5 illustrates a device used in accordance with an aspect of the invention. A cardiac electrogram signal is obtained 510 with a cardiac electrogram sensor on a diaphragm stimulation device. (The sensor may use separate electrode or may use the same electrode that is used for stimulation.) The signal is used by a processor to check cardiac rhythm 520. The processor determines whether or not the rate is high 530. If it is not the device continues to check cardiac rhythm 520. If the rate is high, then the processor determines if the high rate is a tachycardia without ventricular fibrillation or if ventricular fibrillation is occurring 540. Such cardiac electrogram processing techniques are generally known in the art. If ventricular fibrillation is detection, then one or more programmable actions may follow at step 550. For example, sleep apnea therapy comprising diaphragm stimulation may be stopped or controlled to reduce the level of stimulation. The sleep apnea therapy may also be changed to a different therapy such as breathing control where breathing is paced. If at step 540 ventricular fibrillation is not detected, the one or more programmable actions may follow at step 560. For example, the sleep apnea therapy comprising diaphragm stimulation may be stopped or controlled to reduce the level of stimulation. The sleep apnea therapy may also be changed to a different therapy such as deep inspiration which may affect the tachycardia. If a sinus rhythm is not detected as step 570 then cardiac rhythm is checked again at step 520. If a sinus rhythm is detected at step 570, then the appropriate sleep apnea therapy is reinitiated at step 580.

1. A method for treating a patient comprising:
providing an electrical stimulation device configured to provide electrical stimulation to activate a diaphragm to cause contraction;
providing an EGM sensor configured to sense a cardiac rhythm;
sensing a cardiac rhythm and
adjusting the electrical stimulation to the diaphragm based on the cardiac rhythm.

2. The method of claim 1 wherein the step of adjusting the stimulation comprises stopping the stimulation.

3. The method of claim 1 wherein the step of adjusting the stimulation comprises changing a type of electrical stimulation from a first type of stimulation to a second type of stimulation.

4. The method of claim 3 wherein the first type of stimulation comprises a stimulation configured to increase functional residual capacity.

5. The method of claim 3 wherein the second type of stimulation comprises a stimulation configured to elicit an increased tidal volume.

6. The method of claim 1 wherein the step of providing a stimulator comprises providing a stimulator at one of: at the diaphragm, on the phrenic nerve, implanted transvenously, located externally and located subcutaneously.

7. A device for stimulating diaphragm contraction in a subject comprising:
an electrical stimulation device configured to provided electrical stimulation to a subject to activate a diaphragm;
a device interaction controller configured to control interaction of the electrical stimulation device with a cardiac rhythm management device.

8. The device of claim 7 wherein the device interaction controller is configured to detect operation of the cardiac rhythm management device and to adjust electrical stimulation by the electrical stimulation device in response to detecting operation of the cardiac rhythm management device.

9. The device of claim 8 wherein the device interaction controller is configured to turn off electrical stimulation in response to detecting operation of the cardiac rhythm management device.

10. The device of claim 7 wherein the electrical stimulation device is configured to provide electrical stimulation to increase functional residual capacity of a subject.

11. A cardiac rhythm management device in combination with a diaphragm stimulation device comprising:
a cardiac stimulation device configured to provide stimulation to control cardiac rhythm of a subject; and
an electrical stimulation device configured to provided electrical stimulation to a subject to activate a diaphragm to increase functional residual capacity of the subject.

12. The cardiac rhythm management device of claim 11 further comprising:
a device interaction controller configured to control interaction of the electrical stimulation device with the cardiac rhythm management device.
